(19) United States
(12) Patent Application Publication
(10) Pub. No.: US 2016/0317007 A1
(43) Pub. Date: Nov. 3, 2016

(54) VIDEO CAPABLE ELECTRIC TOOTHBRUSH FOR PROPER PERIODONTIUM PROPHYLAXIS

(71) Applicant: Raymond Addington, Atlanta, GA (US)

(72) Inventor: Raymond Addington, Atlanta, GA (US)

(21) Appl. No.: 15/185,246

(22) Filed: Jun. 17, 2016

Related U.S. Application Data
(63) Continuation-in-part of application No. 14/824,124, filed on Aug. 12, 2015.
Application No. 14/824,124 is a continuation-in-part of application No. 14/121,521, filed on Sep. 15, 2014.

(51) Int. Cl. A61B 1/24; A61B 1/00; A61B 1/06; A46B 15/00; A46B 9/04; A46B 5/00; A61C 17/22; A61C 17/40; A61B 1/05; A61B 1/005

(52) U.S. Cl. CPC A61B 1/24; A61B 1/05; A61B 1/00045; A61B 1/00009; A61B 1/0676; A61B 1/0684; A61B 1/00018; A61B 1/005; A46B 9/04; A46B 5/0095; A61C 17/225; A61C 17/40; A61C 17/221; A46B 15/0016

(57) ABSTRACT

A video capable electric toothbrush for proper periodontium prophylaxis having a rotating bristle assembly configured to rotate in multiple directions so as to implement a brushing technique having the brush rotate in a direction from the gum towards the tooth by use of a switch, and regardless of the specific tooth or teeth being brushed, the electric toothbrush is configured to brush along the gingival margin towards the tooth crown. The video capability allows a user to see and target brushing along the gingival margin, and recognize areas with high debris quantity requiring additional attention.

### STATEMENT OF RELATED APPLICATIONS

This patent application is based on and claims the benefit of U.S. patent application Ser. No. 14/824,124 having a filing date of 12 Aug. 2015, which is based on and claims the benefit of U.S. patent application Ser. No. 14/121,521 having a filing date of 15 Sep. 2014.

### BACKGROUND OF THE INVENTION

1. Field of the Invention

The present invention generally relates to an electric toothbrush. More specifically, the present invention relates to a video capable electric toothbrush for proper periodontium prophylaxis, that is, proper prophylaxis of the gums.

2. Prior Art

Commercially available electric toothbrushes have advanced dramatically since the first manual toothbrushes. These electric toothbrushes typically leverage battery-powered motors to rapidly oscillate rotating bristle bundles back-and-forth on a tooth surface, to rapidly vibrate bristle bundles upon a tooth surface, and/or to sweep bristle bundles back-and-forth on a tooth surface, etc. The goals are usually the same: maximizing the rate of bristle-to-tooth scrubbing, minimizing the time and effort necessary for the tooth brusher, and recreating the proper dental prophylaxis procedure at home on a daily basis.

Nonetheless, despite these advancements, current commercially available electric toothbrushes fail to recreate the proper dental prophylaxis procedure. To make matters worse, current commercially available electric toothbrushes actually work against recreating the proper dental prophylaxis procedure as they magnify harmful and counterproductive practices by the tooth brushers.

As context, the proper dental prophylaxis procedure typically involves a three-step process. First, a dental professional carefully scales all plaque and debris off of the exposed surfaces of each tooth, notably, without brushing or scrubbing. Second, the professional buffs the previously scaled surfaces of each tooth. Third, the professional flosses the unexposed surfaces of each tooth.

For the first step, the professional usually employs a specialized manual scaling tool, and/or a rapidly vibrating scaling tool, and/or a water jet often comprising a grit such as baking soda. The scaling tool is configured to scrape and grind the plaque and debris off of the exposed surfaces of each tooth without damaging the tooth enamel. The professional scales the tooth surfaces to the gingival margin of the periodontium (i.e., the gum line), but the professional does not go beyond the gingival margin, for fear of damaging the tissue.

For the second step, the professional usually employs a cup-like buffing tool (shaped to partially envelope the tooth) and a granular buffing medium held by the cup-like portion. The cup-like buffing tool is configured to rapidly rotate the cup with the granular buffing medium upon the previously scaled tooth. In this way, the professional finely buffs and smooths the roughly scaled tooth with the granular buffing medium. Like the first step, the professional buffs the tooth surfaces only to the gingival margin.

For the third step, the professional usually employs floss. The floss is flattened to readily enter the tight spaces between two adjacent teeth. The floss is configured to scrape the plaque and debris off the unexposed surfaces of each tooth without damaging the tooth enamel. The professional uses the floss as a way of initially scaling the unexposed tooth surfaces, and as a way of removing any plaque or debris that might have been trapped in between teeth after the first step. Like the first and second steps, the professional flosses the tooth surfaces only to the gingival margin.

If the gingival margin is loosened or receded, the professional usually avoids scaling, buffing, or flossing the tooth surfaces beyond where the gingival margin should be. The professional understands that continued trauma (e.g., tissue damage, infection, inflammation) to the gingival margin results in a worsening condition. Moreover, the professional understands that the nearly exposed tooth dentin (due to a lack of tooth enamel where the gingival margin should be) is extremely sensitive and painful to work on. As such, the professional usually rinses and wipes and/or picks-away any plaque or debris caught beyond the gingival margin. Sometimes, the professional specialty treats the area if the situation is severe.

Within that context, the proper dental prophylaxis procedure has three main focuses: removing plaque and debris from the surfaces of the tooth, protecting and preserving the tooth enamel, and protecting and preserving the gingival margin. Unfortunately, even though prior art electric toothbrushes are well equipped to power scrub debris from the tooth enamel, they can be devastating over time to the gingival margin.

More specifically, daily home tooth brushing involves bristles, which are an effective, efficient, and affordable system for clearing debris from the tooth enamel before it becomes hardened plaque. However, regardless of the “softness” of the bristle bundles, or the brushing technique and/or angle employed by the tooth brusher (the Bass method, for example), prior art electric toothbrushes inherently brush beyond and against the junction of the tooth and the periodontium. The rapidly oscillating and rotating bristle bundles (designed to imitate the professional cup-like buffing tool), the rapidly vibrating bristle bundles, the back-and-forth sweeping bristle bundles, etc. (intended to maximize the rate of bristle-to-tooth scrubbing) unintentionally force bristles into the junction. Over time, tissue damage along the gingival margin results in lateral movement of the periodontium away from the tooth cementum-layer upon which it is anchored (a key indication of periodontium disease).

There is, therefore, a need in the art for an electric toothbrush for proper periodontium prophylaxis. It is to this need that the present invention is directed. Accordingly, the present invention provides an improved electric toothbrush effectively overcoming the aforementioned difficulties and longstanding problems inherent in the art. These problems have been solved in a simple, efficient, and highly effective way by which to construct an electric toothbrush.

### BRIEF SUMMARY OF THE INVENTION

Briefly, the present invention is an electric toothbrush for proper periodontium prophylaxis. In one illustrative embodiment, the electric toothbrush includes a handle body, an interchangeable stem, and a camera device with intraoral video capability. The interchangeable stem includes an elongate neck, a head, a rotating bristle assembly, and a means for rotating the rotating bristle assembly. In another illustrative embodiment, the electric toothbrush includes a unitary handle and stem.

The stem preferably comprises a rotating bristle assembly mounted on an elongate neck, a bristle guard that acts as a mouth or cheek shield for covering a portion of the rotating bristle assembly, a means for mounting the rotating bristle assembly to the stem (in the interchangeable stem embodiment), a means for rotating the rotating bristle assembly, a means for mounting the stem to the handle body, and a means for connecting the rotating bristle assembly to a motor for rotating the rotating bristle assembly.

The elongate neck has a first end opposite a second end. The first end of the elongate neck is configured to detachably engage with the handle body, and comprises the means for mounting the stem to the handle body. The second end of the elongate neck is terminated by the head, and comprises the rotating bristle body and the mouth or cheek shield.

The head comprises a generally rotating bristle assembly longitudinally aligned with the longitudinal axis of the interchangeable stem. The rotating bristle assembly has a first end opposite a second end. The first end of the rotating bristle assembly is proximate to the second end of the elongate neck. The bristle guard extends from the second end of the neck around the side of the rotating bristle assembly. Extending from the second end of the cylindrical bristle body is the means for rotating the rotating bristle assembly, such as, for example, a metal or plastic rod or wire, extending through the elongate neck towards the handle body. The means for rotating the rotating bristle assembly cooperates with the motor for rotating the rotating bristle assembly when the motor is activated.

The bristle guard is attached to or part of the stem proximal to the first end of the elongate neck and proximal to the rotating bristle assembly, and extends circumferentially around and laterally spaced apart from the rotating bristle assembly. The bristle guard describes an arc segment along the rotating bristle assembly. The longitudinal axis of the bristle guard is parallel to the longitudinal axis of the interchangeable stem. The bristle guard acts to prevent the rotating bristle assembly from contacting the inner cheek of the user when the device is being operated.

The handle body preferably comprises a cylindrical portion having a hollow interior within which the motor and the power source, and appropriate wiring and electrical connectivity and operating means, are located. The handle body further comprises a means for detachably engaging or rigidly/permanently engaging (alternatives) the camera device to an exterior side of the cylindrical portion such that intraoral video may be obtained during operation of the electric toothbrush. The camera device may, alternatively, be detachably engaged to the exterior side of the elongate neck.

Furthermore, the handle body also comprises a means for mounting the stem to the rotating bristle assembly (in the interchangeable stem embodiment), a means for accessing the interior of the handle body, and a switch means for activating and deactivating (turning on and off) the motor. In certain illustrative embodiments, the interior of the handle body is configured to hold rechargeable batteries, the motor, electronic boards and circuitry, system bus, etc. so as to operate/regulate/control the rotating bristle assembly and/or capture/process/transmit data captured by the camera device. In other illustrative embodiments, the interior of the handle body is configured to hold exchangeable batteries.

Preferably, the motor is configured such that the rotating bristle assembly can rotate in both directions, such as clockwise and counterclockwise, as the user desires. Moreover, the camera device is configured for 640×480 resolution or upwards of 4K HD video or any other video capture capability known to a person having ordinary skill in the art. Moreover, the camera device may be an optical device consisting of a rigid or flexible tube, and/or cylindrical optical/conductive cable, and/or any communications pathway, with a lens on one end, and a camera sensor on the other end linked together by a relay optical system there between. The camera device, when detachably engaged to the handle body, or when permanently and rigidly attached, may aim its aperture towards the oral cavity of a user of the electric toothbrush so as to capture intraoral video. The intraoral video may then be transmitted and/or processed for useful display to the user of the electric toothbrush

In the interchangeable stem embodiment, the means for rotating the rotating bristle assembly is detachably and operably connectable to the motor or to an extension of the motor, whereby when the motor is activated, the motor turns the means for rotating the rotating bristle assembly, thus rotating the rotating bristle assembly. In the non-interchangeable stem embodiment, the means for rotating the rotating bristle assembly is operably connected to the motor or to an extension of the motor, whereby when the motor is activated, the motor similarly turns the means for rotating the rotating bristle assembly, thus rotating the rotating bristle assembly.

In both the interchangeable and the non-interchangeable stem embodiments, the motor preferably can rotate the rotating bristle assembly in both directions; therefore, the device further comprises a switch that can cause the motor to rotate in both directions. Alternatively, the device can comprise appropriate gearing so as to accomplish the same result of allowing the rotating bristle assembly to rotate in both directions. Thus, when the means for rotating the rotating bristle assembly is powered, the rotating bristle assembly is selectably motorized to rotate clockwise or counterclockwise via mechanical engagement with the means for rotating the rotating bristle assembly.

In another illustrative embodiment, the bristle guard of the interchangeable stem comprises a concave bristle-facing side and a convex non-bristle facing side. The non-bristle facing side can define or comprise a raised surface texture or raised surface feature that is abrasive or frictional and that can be used for tongue, cheek, and gum cleaning and/or massaging.

These features, and other features and advantages of the present invention will become more apparent to those of ordinary skill in the relevant art when the following detailed description of the preferred embodiments is read in conjunction with the appended drawings in which like reference numerals represent like components throughout the several views.

### BRIEF DESCRIPTION OF THE DRAWINGS

FIG. 1A is a front side perspective view of an embodiment of an electric toothbrush of the present invention.

FIG. 1B is a right side perspective view of the electric toothbrush of FIG. 1A turned 90 degrees.

FIG. 2A is a top view of the electric toothbrush of FIG. 1A.

FIG. 2B is a bottom view of the electric toothbrush of FIG. 1A.

FIG. 3 is a partially exploded perspective view of the electric toothbrush of FIG. 1 illustrating how an interchangeable stem and a handle body detach and attach to one another.

FIG. 4A is an enlarged front perspective view of the interchangeable stem of FIG. 3.

FIG. 4B is a cross section of the interchangeable stem of FIG. 4A.

FIG. 4C is a second embodiment of the interchangeable stem of an electric toothbrush of the present invention.

FIG. 5A is a cross-sectional view of the handle body of FIG. 3.

FIG. 5B is the cross-sectional view of FIG. 5A turned 90 degrees.

FIG. 6 is a partial perspective view of the electric toothbrush of FIG. 1 operating on the outside facing surfaces of teeth on the upper right side, or the lower left side, of a user's jaw.

FIG. 7 is a partial perspective view of the electric toothbrush of FIG. 1 operating on the outside facing surfaces of teeth on the upper left side, or the lower right side, of a user's jaw.

FIG. 8 is a partial perspective view of the electric toothbrush of FIG. 1 operating on the inside facing surfaces of teeth on the upper right side, or the lower left side, of a user's jaw.

FIG. 9 is a partial perspective view of the electric toothbrush of FIG. 1 operating on the inside facing surfaces of teeth on the upper left side, or the lower right side, of a user's jaw.

FIG. 10A is a front side perspective view of an electric toothbrush of the present invention including a second embodiment of the handle and a second embodiment of the bristle shield.

FIG. 10B is a right side perspective view of the electric toothbrush of FIG. 10A turned 90 degrees.

FIG. 11A is a front side perspective view of a first embodiment of a video capable electric toothbrush of the present invention.

FIG. 11B is a partial perspective view of the electric toothbrush of FIG. 11A operating on the outside facing surfaces of teeth on the upper right side, or the lower left side, of a user's jaw.

FIG. 11C is a partial perspective view of the electric toothbrush of FIG. 11A operating on the outside facing surfaces of teeth on the upper left side, or the lower right side, of a user's jaw.

FIG. 12A is a front side perspective view of a second embodiment of a video capable electric toothbrush of the present invention.

FIG. 12B is a partial perspective view of the electric toothbrush of FIG. 12A operating on the outside facing surfaces of teeth on the upper right side, or the lower left side, of a user's jaw.

FIG. 12C is a partial perspective view of the electric toothbrush of FIG. 11A operating on the outside facing surfaces of teeth on the upper left side, or the lower right side, of a user's jaw.

### DETAILED DESCRIPTION OF PREFERRED EMBODIMENTS

Preferred embodiments and aspects of the present disclosure provide an electric toothbrush for proper periodontium prophylaxis. Unlike prior art electric toothbrushes, the electric toothbrush of the present disclosure is configured to effectively and efficiently remove plaque and debris from the tooth enamel and to protect and preserve the gingival margin. The electric toothbrush maximizes the rate of bristle-to-tooth scrubbing, minimizes the time and effort necessary for the tooth brusher, and recreates the proper dental prophylaxis procedure at home on a daily basis. Moreover, embodiments of the electric toothbrush may be outfitted with an intraoral video camera device.

More specifically, by a simple flip of a switch, and regardless of the specific tooth or teeth being brushed, the electric toothbrush is configured to brush along the gingival margin towards the tooth crown. The electric toothbrush, thereby, prevents the bristles from being forced improperly against the junction of the tooth and the periodontium. As such, the electric toothbrush clears debris away from the tooth enamel (before it becomes hardened plague) without sacrificing the gingival margin.

Furthermore, the electric toothbrush facilitates appropriate daily prophylaxis by steering the tooth brusher away from harmful behavior, and by affording the very young, the elderly, the infirm, and the lazy with an affordable tool that requires not only one hand, but minimal hand, wrist, and arm motion to operate. Moreover, embodiments of the electric toothbrush may have a bristle shield that extends around the bristle structure of the electric toothbrush. The bristle shield is configured to: shield the bristles from over-the-air contamination, when it is in a “full” bathroom environment, and to facilitate US American Dental Association (ADA)-recommended drying of the bristles between brushings.

Furthermore, embodiments of the electric toothbrush may comprise a camera device with intraoral video capability, as well as a power source, wiring, electronic boards/circuitry, system bus, and/or means for transmitting video data for use, directly or indirectly, by a user. In this way, the electric toothbrush may be further configured to facilitate appropriate daily prophylaxis because the intraoral video capability allows the user to see and target brushing along the gingival margin, and recognize areas with high debris quantity requiring additional attention. Because the appropriate daily prophylaxis accomplished by the electric toothbrush of the present invention requires minimal toothpaste (and the foam created therefrom), a video capable embodiment is not limited by obstructed video imaging. This would be the outcome of merely sticking a video camera on the side of a traditional prior art toothbrush that encourages harmful prophylaxis.

Furthermore, embodiments of the electric toothbrush comprise an interchangeable stem and an attachment means for allowing the attachment of the interchangeable stem to a handle body. The interchangeable stem includes, at one end, a toothbrush head with a rotating bristle assembly and a neck for attachment via the attachment means to the handle body. The rotation means utilized to allow the rotation of the interchangeable stem may be a component of the stem or the handle body depending on the embodiment. It is, however, preferred that the rotation means be a part of the handle body in order to reduce the manufacturing costs of the interchangeable stems. This also allows for the stems to be marketed relatively inexpensively and independently of the handle body.

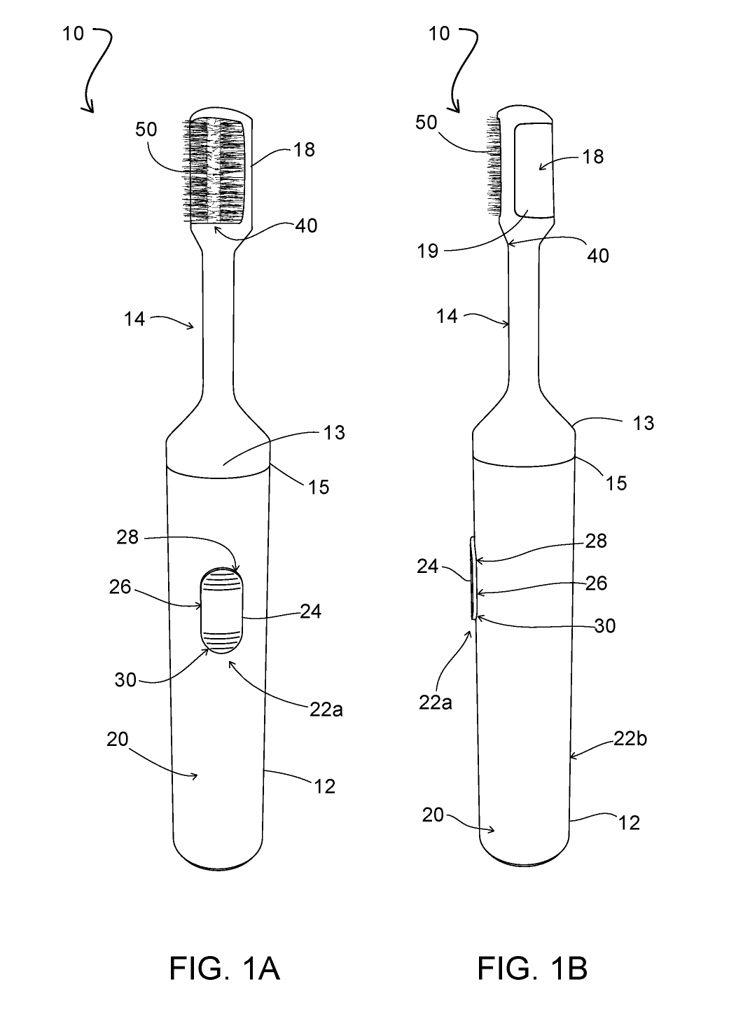

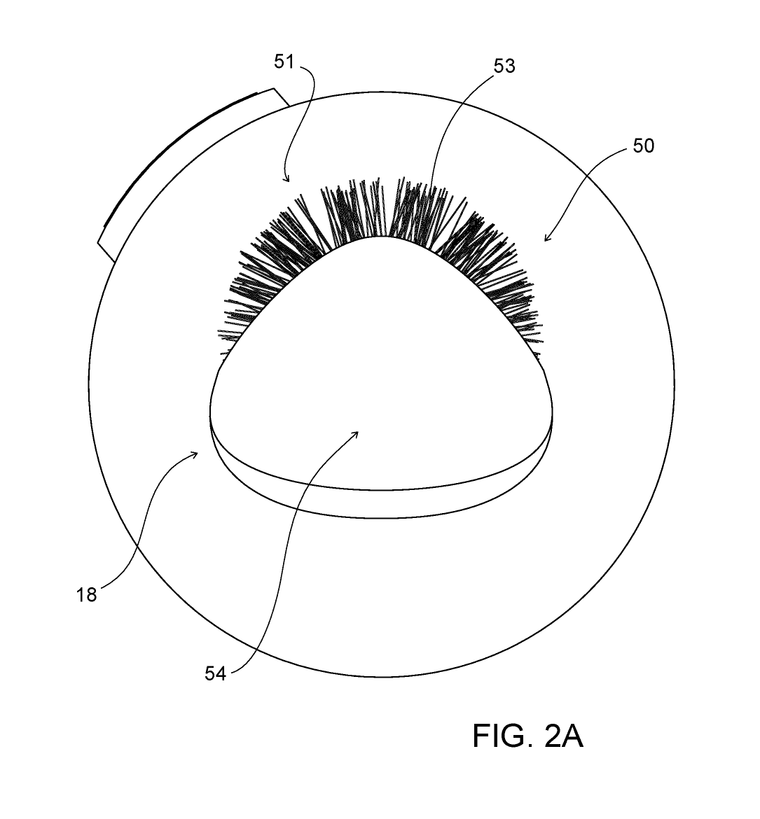

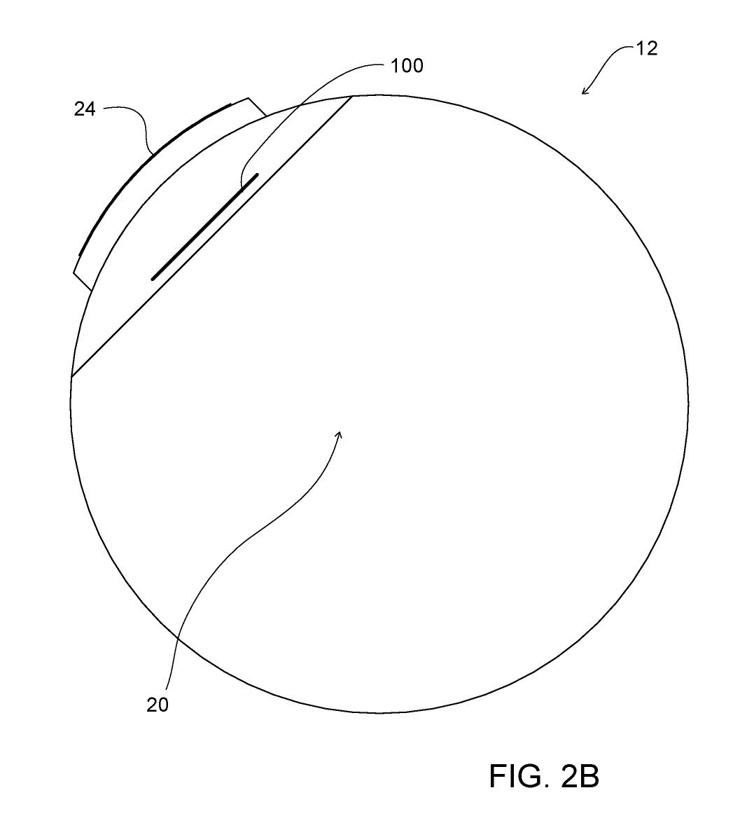

Referring now to the drawings, wherein the showings are for purposes of illustrating the various embodiments of the present disclosure only, and not for purposes of limiting the same, FIGS. 1A-1B and 2A-2B show the general features of the present invention. FIG. 1A is a front side perspective view of an embodiment of an electric toothbrush of the present invention, FIG. 1B is a right side perspective view of the electric toothbrush of FIG. 1A turned 90 degrees, FIG. 2A is a top view of the electric toothbrush of FIG. 1A, and FIG. 2B is a bottom view of the electric toothbrush of FIG. 1A.

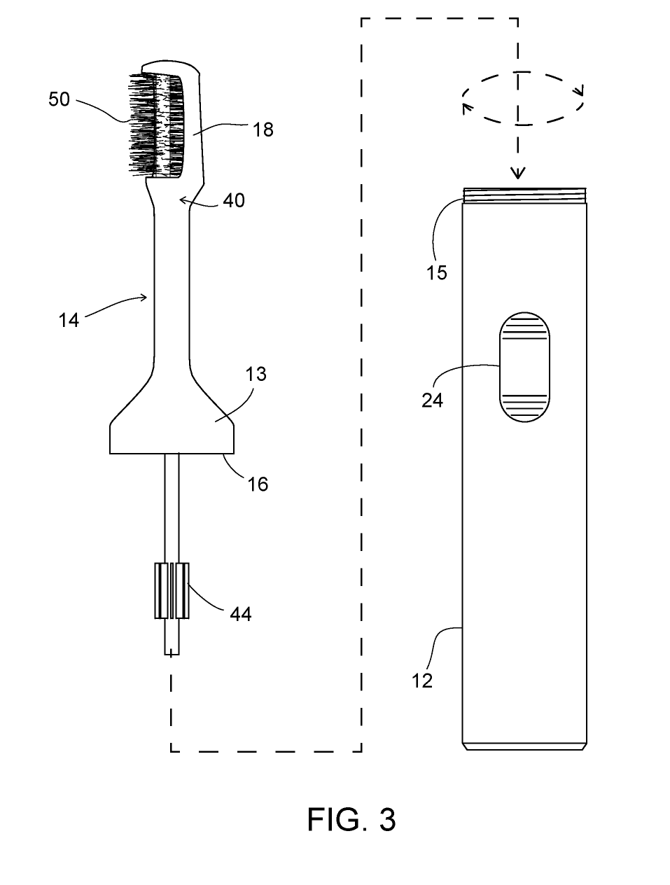

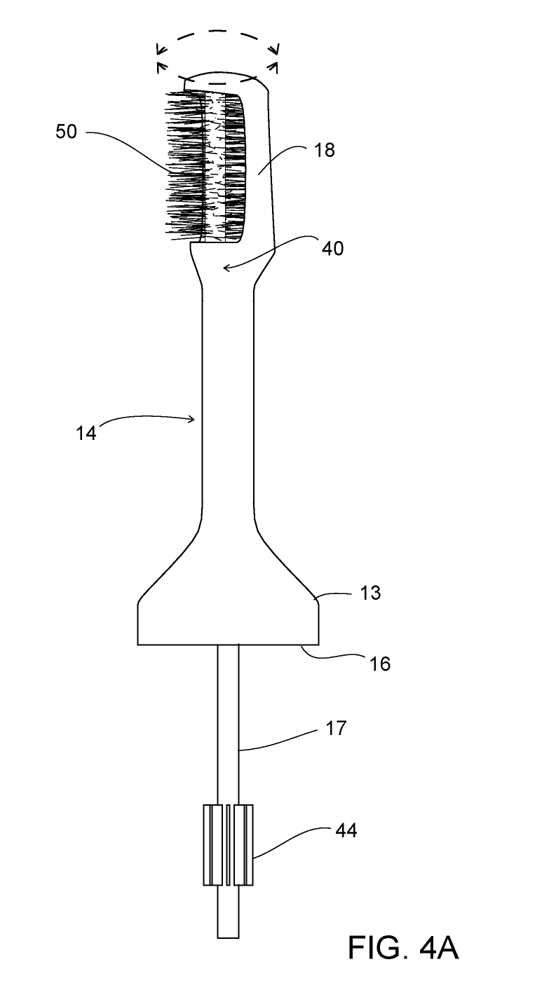

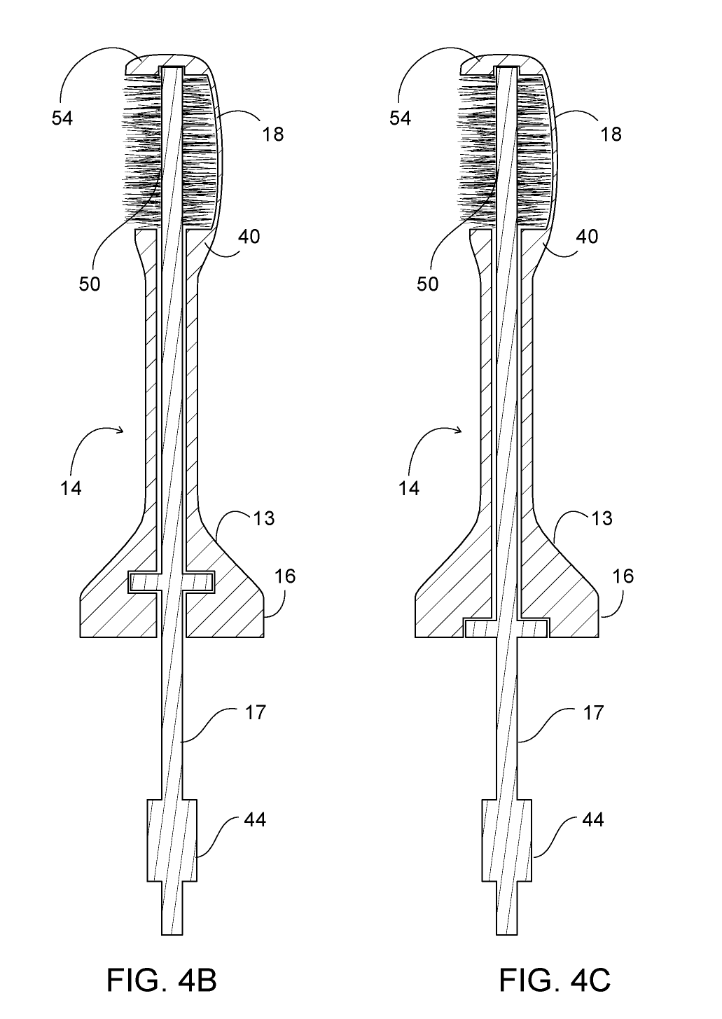

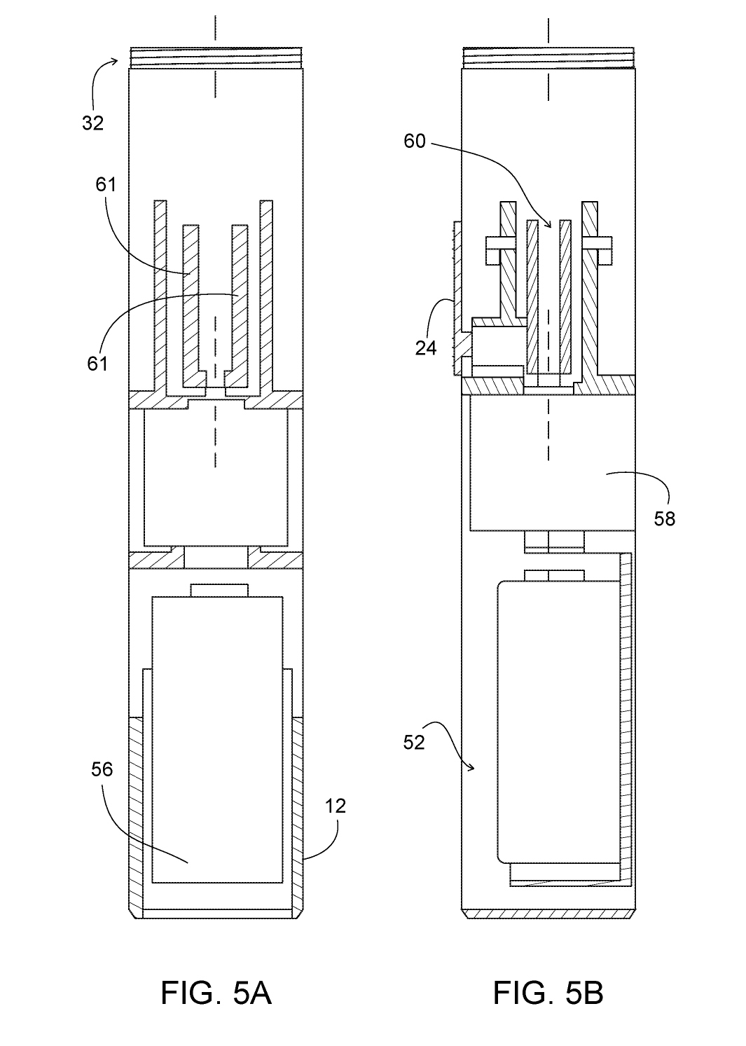

FIGS. 3, 4A-4C, and 5A-5B show the additional general features of an interchangeable stem embodiment of the invention. FIG. 3 is a partially exploded perspective view of the electric toothbrush of FIG. 1 illustrating how an interchangeable stem and a handle body detach and attach to one another. FIG. 4A is an enlarged front perspective view of the interchangeable stem of FIG. 3. FIG. 4B is a cross section of the interchangeable stem of FIG. 4A. FIG. 4C is a second embodiment of the interchangeable stem of an electric toothbrush of the present invention. FIG. 5A is a cross-sectional view of the handle body of FIG. 3 and FIG. 5B is the cross-sectional view of FIG. 5A turned 90 degrees.

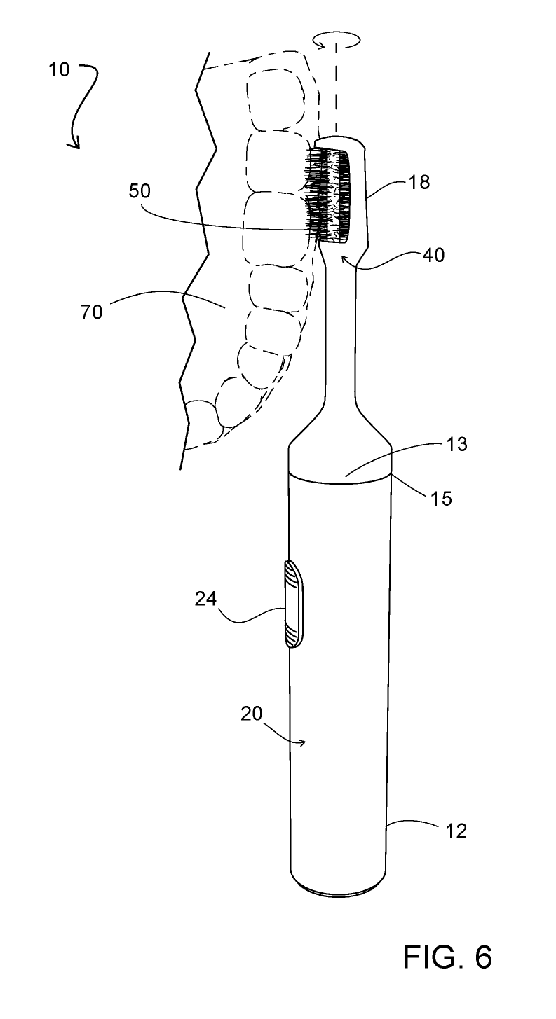

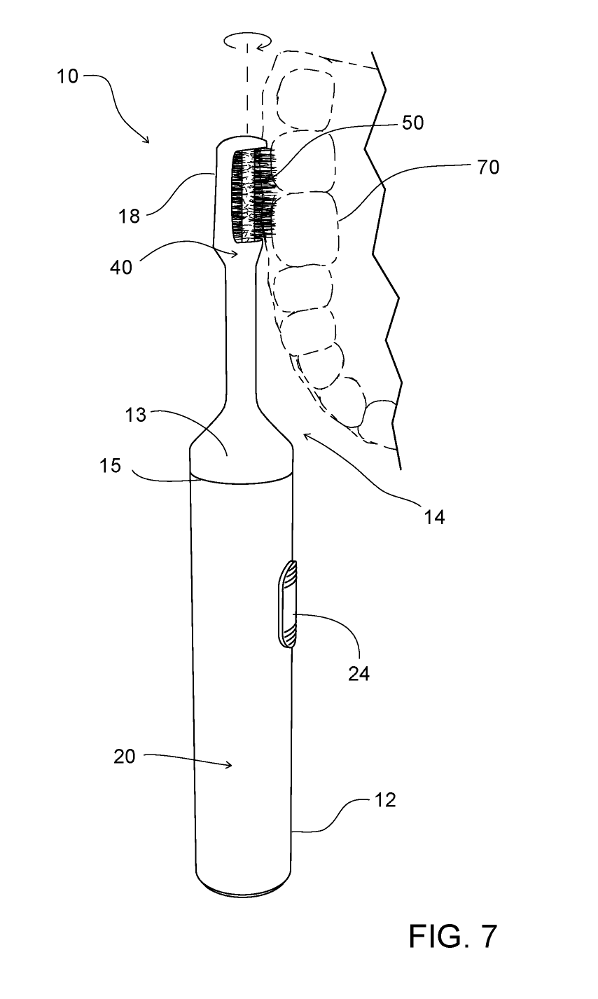

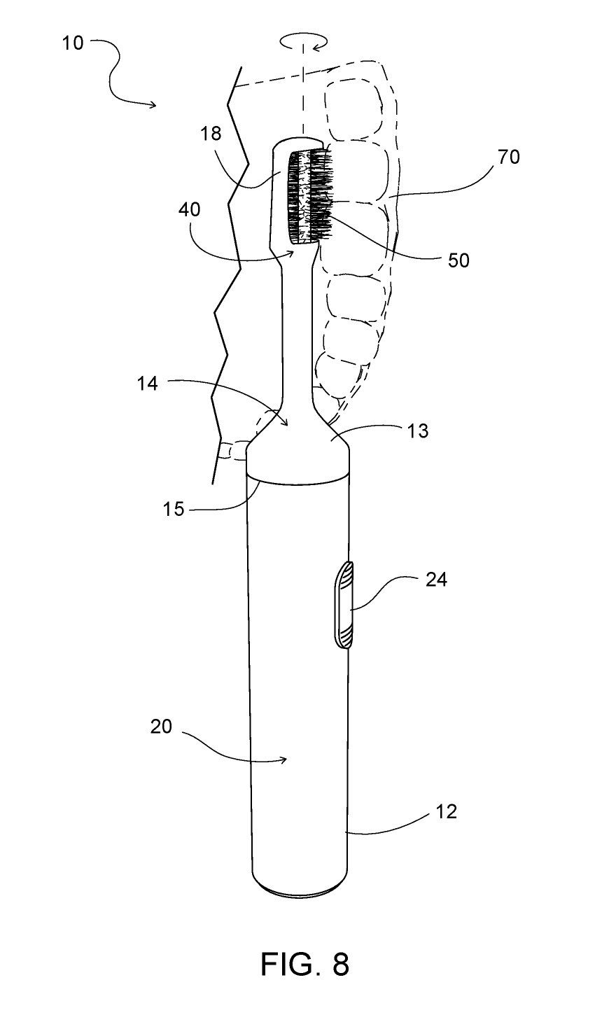

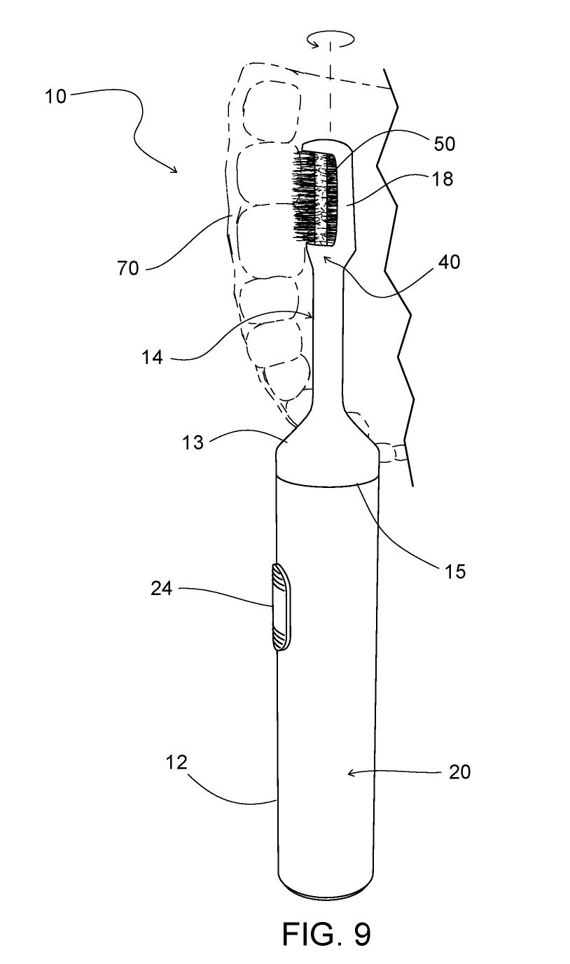

FIGS. 6-9 show an embodiment of the present invention in use. FIG. 6 is a partial perspective view of the electric toothbrush of FIG. 1 operating on the outside facing surfaces of teeth on the upper right side, or the lower left side, of a user's jaw. FIG. 7 is a partial perspective view of the electric toothbrush of FIG. 1 operating on the outside facing surfaces of teeth on the upper left side, or the lower right side, of a user's jaw. FIG. 8 is a partial perspective view of the electric toothbrush of FIG. 1 operating on the inside facing surfaces of teeth on the upper right side, or the lower left side, of a user's jaw. FIG. 9 is a partial perspective view of the electric toothbrush of FIG. 1 operating on the inside facing surfaces of teeth on the upper left side, or the lower right side, of a user's jaw.

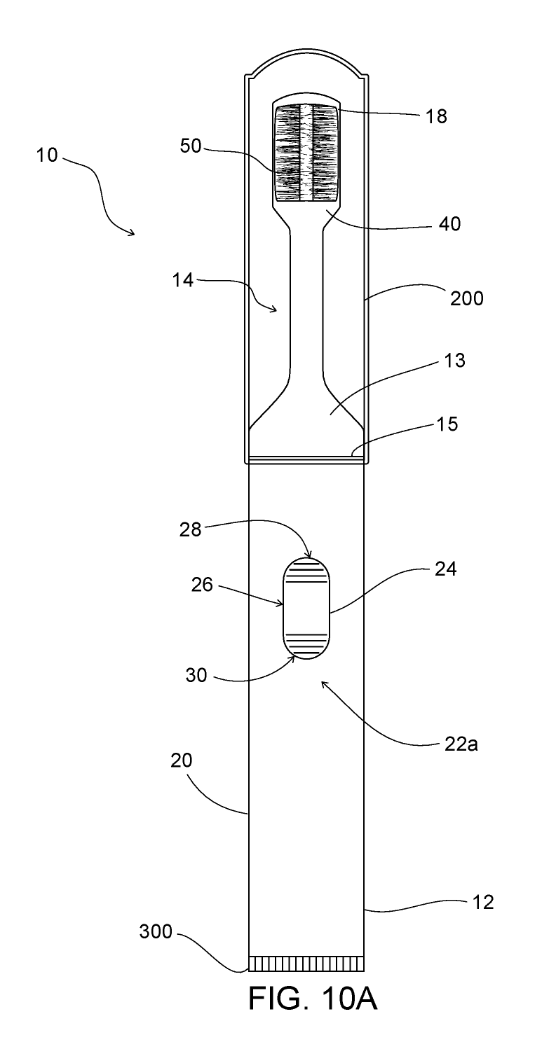

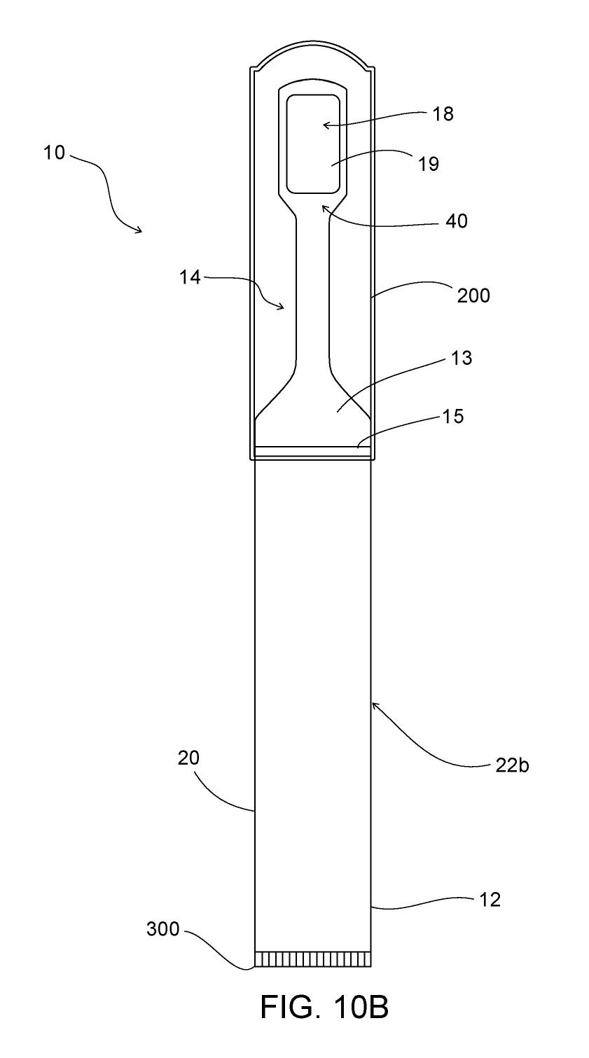

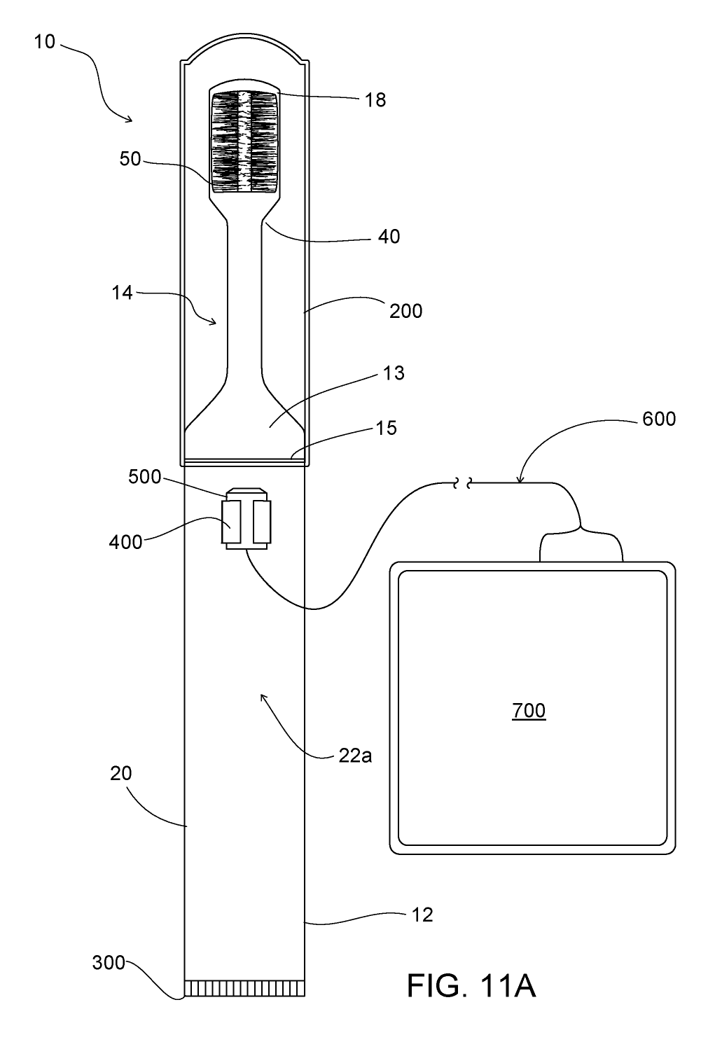

FIG. 10A is a front side perspective view of an electric toothbrush of the present invention including a second embodiment of the handle and a second embodiment of the bristle shield. FIG. 10B is a right side perspective view of the electric toothbrush of FIG. 10A turned 90 degrees.

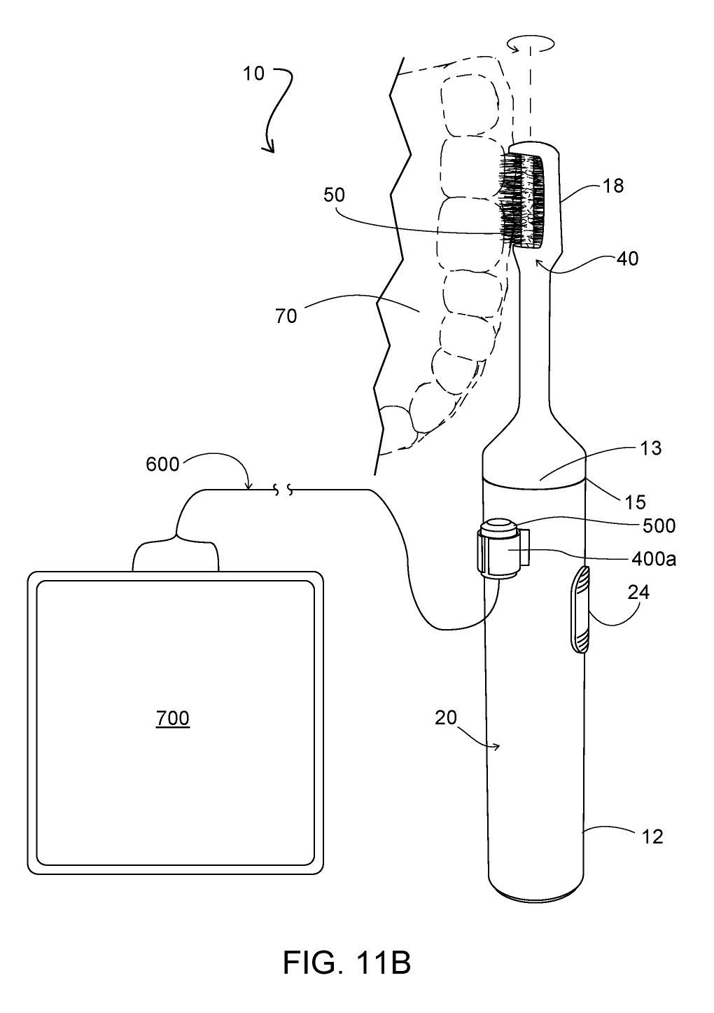

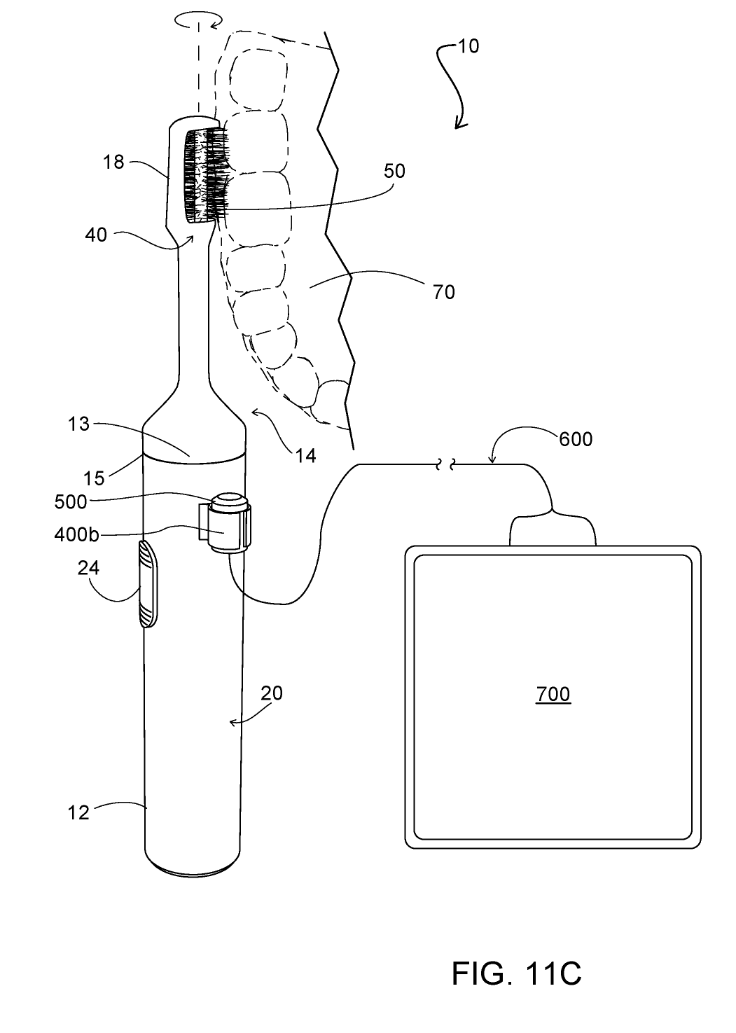

FIG. 11A is a front side perspective view of a first embodiment of a video capable electric toothbrush of the present invention. FIG. 11B is a partial perspective view of the electric toothbrush of FIG. 11A operating on the outside facing surfaces of teeth on the upper right side, or the lower left side, of a user's jaw. FIG. 11C is a partial perspective view of the electric toothbrush of FIG. 11A operating on the outside facing surfaces of teeth on the upper left side, or the lower right side, of a user's jaw.

Referring now to FIGS. 1A-1B, 2A-2B, 3, and 4A-4C, one embodiment of the electric toothbrush for proper periodontium prophylaxis, toothbrush 10, comprises a handle body 12 (also referred to as just the handle 12) and an interchangeable stem 14 (also referred to as just the stem 14) having a head 40 with a rotating bristle assembly 50. The handle body 12 comprises the motor 58, battery 56, and electrical components for powering the rotating bristle assembly 50, and for providing a suitable handgrip. The interchangeable stem 14 comprises the rotating bristle assembly 50 for brushing the teeth.

Another embodiment of the toothbrush 10 is a unitary device in which the stem 14 is attached, permanently, to the handle 12. Also in this embodiment, the handle 12 comprises the motor 58, battery 56, and electrical components for powering the rotating bristle assembly 50, and for providing a suitable handgrip, and the stem 14 comprises the rotating bristle assembly 50 for brushing the teeth.

The handle 12 comprises an ergonomic outer surface 20, and an internal cavity 52 for containing a rotation means. The rotation means comprises a battery 56, a motor 58, and a set of electrical and transmission components. The handle body 12 may have a larger diameter than a standard, prior art electric toothbrush, which makes handle 12 more comfortable and easier to use as less force is required to stably hold toothbrush 10. Also, when handle 12 is larger, the toothbrush 10 requires less rotational turning about its longitudinal axis than is required by a smaller diameter handle.

Furthermore, the handle 12 is preferably generally cylindrical in shape and has a cross section that is predominantly circular, preferably between about one-half inch to one and one-half inches in diameter, and more preferably approximately one inch in diameter. Handle 12 further may comprise a generally flat portion 22 defined by a chord of the circular cross section of the handle 12. Along the surface of the flat portion 22, the handle 12 comprises an activator slide 24 (a switch) configured to turn the electric toothbrush 10 “on” and “off” and to control the rotational direction (clockwise or counterclockwise) of the rotating bristle assembly 50 during operation (described in greater detail below). Therefore, the handle 12 may be oversized with the front flat surface 22 to enhance manual dexterity, and to make gripping more comfortable for the user. Alternatively, the handle 12 may be completely cylindrical, or have an oval or other geometric cross section, and the activator slide 24 may be simply mounted on or through the wall of the handle 12.

Activator slide 24 preferably slides within a channel 26. When the activator slide 24 is in a first position (i.e., when activator slide 24 is fully slid towards a first end 28 of the channel 26), the electric toothbrush 10 is in a first “on” position and the cylindrical bristles bundle 50 rotate clockwise (power is provided to the motor 58 causing rotation of the rotating bristle assembly 50). When activator slide 24 is in a second position (i.e., when activator slide 24 is fully slid towards a second end 30 of the channel 26), the electric toothbrush 10 is in a second “on” position and the cylindrical bristles bundle 50 rotate counterclockwise (power is provided to the motor 58 causing rotation of the rotating bristle assembly 50). When activator slide 24 is in an intermediate position (i.e., when activator slide 24 is slid to an intermediate position between the first end 28 and the second end 30 of the channel 26), the electric toothbrush 10 is an “off” (power is not provided to the motor 58). It is, of course, within the scope of the invention to use alternate means of switching the electric toothbrush 10 from “on” to “off”, etc., such as a water-proof single press button with various stages, or a plurality of water proof individual push buttons with independent and discrete functions.

The outer surface 20 of the handle 12 may be formed of, or comprised of, a “squeezable” or compressible material that deflects slightly when a gripping force is applied. Such a construction makes the toothbrush 10 more comfortable to hold and, therefore, easier to use. Optionally, the handle 12 may be formed in a double-sided configuration with an elongate flat portion 22b antipodal to, or at any other angle relative to, the flat portion 22a.

Alternatively, the outer surface 20 of the handle 12 also may comprise grips, ridges, bumps, surface features, and/or surface textures (not depicted in the FIGS.) to facilitate a user firmly grasping the handle 12, particularly, when the handle 12 is wet. Further preferred enhancements include grips, surface features, and/or surface textures that are aesthetically pleasing in appearance, although any number, shape, structure, and/or configuration may be included. As such, the provision of the outer surface 20 allows the user of the electric toothbrush 10 to appropriately brush the periodontium, and all the tooth surfaces, while essentially maintaining the same grip on the handle 12. Simple adjustments are possible for the user via slight pronation of the wrist and a switch in the rotational direction of the rotating bristle assembly 50.

A rotating ring 15 can be attached to the top portion of the handle 12 as an illustrative attachment mechanism between the stem 14 and the handle 12. For example, the rotating ring 15 is configured to detachably receive the stem 14 at the end opposite the head 40. The rotating ring 15 preferably has a fixed portion which is secured to the handle 12, and a rotating portion designed to be turned 360 degrees in either a clockwise or counterclockwise direction. The rotating ring 15 has a threaded inner surface 32 (best seen in FIGS. 5A and 5B) complementary of a corresponding threaded outer surface 16 on the stem 14, at the end opposite the head 40 (best seen in FIGS. 4A-4B). The rotating ring 15, therefore, is configured to thread the stem 14 onto the handle 12 (best seen in FIG. 3) such that the stem is rigidly retained by the handle 12, and such that stem 14 is mechanically engaged with the rotation means of the electric toothbrush 10 (described in greater detail herein). It is envisioned that the rotating ring 15 may be a part of the stem 14 and, therefore, the system described would be reversed relative to the handle 12 and the stem 14. Alternatively, another attachment mechanism between the stem 14 and the handle 12 can be a snap fit or cooperating clips.

The handle 12 comprises an internal cavity 52 (best seen in FIGS. 5A and 5B) that houses a rotation means configured to drive rotation of the rotating bristle assembly 50. The rotation means includes a battery 56 and a motor 58 that the battery 56 powers. The battery 56 is accessible from a snap lid 100 on the bottom of the handle 12 (best seen in FIG. 2B). The rotation means further includes a drive axle 60, which is operatively connected to and powered by motor 58 on a first side, and a splined cylindrical mandrel 61, which is attached to and rotated about its longitudinal axis by the motor 58 rotating the drive axle 60. The splined cylindrical mandrel 61 is on the side opposite the drive axle 60. The inner surface 60 of the splined cylindrical mandrel 61 defines a series of complementary splines configured to mechanically engage with a portion of the stem 14. As such, when the stem 14 is attached to the handle 12, and when the motor 58 rotates the drive axle 60, the cylindrical mandrel 61 also rotates and transmits the rotation through the stem 14 to the rotating bristle assembly 50.

It is envisioned that various other configurations and structures are possible for the rotation means of the electric toothbrush 10. For example, a clutch configuration or other frictional transmission configuration, a tongue and groove configuration, a hex key and slot configuration all are suitable alternatives. For another example, in the non-interchangeable stem configuration, the drive axle 60 can connect directly to the bristle axle 17 and/or the drive axle 60 and the bristle axle 17 can be the same component extending from the motor 58 to the rotating bristle assembly 50.

Referring now to FIGS. 4A and 4B, the interchangeable stem 14 comprises, at one end, a head 40 with a rotating bristle assembly 50 and a bristle guard 18, and, extending down from the head 40, a neck 40 (best seen in FIGS. 1A and 1B). The neck 40 is elongate so as to allow the head 40 to extend deep into a user's mouth. The neck 40 is terminated in this particular embodiment with the threaded outer surface 16 configured, at least in part, to cooperate with the rotating ring 15 as the attachment means to the handle 12. Generally speaking, the non-interchangeable stem 14 has a configuration analogous to the interchangeable stem 14, with the main exception that the non-interchangeable stem 14 does not have a rotation disc 13 or a threaded outer surface 16, the elongate neck 14 simply being permanently attached to the top of the handle 12.

The neck 40 of the stem 14 is generally a hollow cylinder having a generally cylindrical outer wall 82 defining an elongate cylindrical passage 84 therethrough extending from the base proximal to the handle 12 to the bristle guard 18. The elongate cylindrical passage 84 extends from the bottom of the neck 40 (where the complementary threading 16 is situated) to the top of the neck 40 (where the rotating bristle assembly 50 and the bristle guard 18 are situated). A bristle axle 17 extends through the elongate cylindrical passage 84 of the neck 40 and engages with the rotating bristle assembly 50 such that when the bristle axle 17 is rotated about its longitudinal axis by the rotation means of the handle 14, the rotating bristle assembly 50 also rotates about its longitudinal axis (described in greater detail herein). Depending on the direction of rotation of the rotations means, the rotating bristle assembly 50 is configured to rotate clockwise or counterclockwise.

Depending on the complexity of the rotation means, the rotating bristle assembly 50 of the stem 14 also may be configured to rotate about a fixed or variable axis. For example, as the rotating bristle assembly 50 rotates, the rotating bristle assembly 50 may move towards or away from the longitudinal axis of the toothbrush 10. As such, not only is the rotating bristle assembly 50 configured to brush, sweep, and swipe, clockwise or counterclockwise, in a direction about the longitudinal axis of the toothbrush 10, the rotating bristle assembly 50 also may be configured to variably press harder or softer against a stationary adjacent oral surface during operation.

The bristle guard 18 of the stem 14 extends from above one end of the rotating bristle assembly 50, namely the top end 86, partially over the extended bristles, to the junction of the head 40 with the neck 40 (best seen in FIGS. 1B and 4A-4B). The bristle guard 18 is contiguous with the outer cylindrical wall 82 of the neck 40 (i.e., they substantially share a contiguous outer surface, but are not necessarily co-cylindrical). At the end of the bristle guard 18 opposite the neck 40, the bristle axle 17, which is extending through the rotating bristle assembly 50, engages to and rotatably pivots relative to the bristle guard 18. As such, the bristle guard 18 is not merely hanging over the rotating bristle assembly 50, but instead the bristle guard 18 is retained in place by the bristle axle 17, which is journaled into the top end 86 of the bristle guard 18. Therefore, when the rotating bristle assembly 50 is rotating, the rotating bristle assembly 50 is bounded, at least partially, by the bristle guard 18 above (best seen in FIGS. 4A-4B), by the bristle guard 18 to the side, and by the neck 40 below.

It is envisioned that the width of the bristle guard 18 may take various dimensions; however, it is preferred that the width of the bristle guard 18 extends from a quarter to half-way around the rotating bristle assembly 50. Additionally, it is envisioned that the structure of the bristle guard 18 may take more complex configurations for additional functionality. Moreover, the outer surface 19 of the bristle guard 18 may comprise a raised surface texture and/or surface feature. The outer surface 19 of the bristle guard 18, therefore, may be configured to provide a firm textured surface with which a user may clean or massage or stimulate his or her tongue, cheeks, and/or gums. Of course, the outer surface 19 of the bristle guard 18 may be used as a scaling tool for more abrasive cleaning of an oral surface, should the rotating bristle assembly 50 not provide enough power.

In one embodiment, the outer cylindrical wall 82 of the neck 40 of the stem 14, along with the bristle guard 18 contiguous thereto, may be incrementally rotatable. Said another way, when the stem 14 is attached to the handle 12, the outer cylindrical wall 82 of the neck 40 with the bristle guard 18 may be rotatable about the longitudinal axis of the toothbrush 10. Therefore, the alignment of the bristle guard 18, relative to any point along the outer circumference of the handle 12, is variable. As such, the bristle guard 18 can be configured to rotate in increments around the rotating bristle assembly 50 so as to change whether the bristle guard 18 is on the right side of the rotating bristle assembly 50, whether the bristle guard 18 is aligned with the activator slide 24, whether the bristle guard 18 is antipodal relative to the activator slide 24, or whether the bristle guard 18 is on the left side of the rotating bristle assembly 50, etc.

More specifically, at the end of the neck 40 of the stem 14, above the complementary threaded outer portion 16, is a rotation disk 13. The rotation disk 13 is rigidly engaged with the cylindrical outer wall of the neck 40. The rotation disk 13 is configured to receive one end of the cylindrical outer wall 82 of the neck 40. The cylindrical outer wall 82 rotates against the rotation disk 13 and, with the help of small flexible projections (off of one end of the cylindrical outer layer and not shown) and correspondingly-sized dimples (along the rotation disk 13 and not shown), the rotation is incremental.

The small flexible projections press fit into the correspondingly sized dimples to lock an orientation in place but, with sufficient force, a user can overcome the press-fit to rotate the small flexible projections to the next dimple. Thereby, the user can adjust the alignment of the cylindrical outer wall 82 of the neck 40, and therefore the bristle guard 18, relative to the rotating bristle assembly 50. In a preferred embodiment, the bristle guard 18 may be manually rotated in 10 degree increments. However, it will be clear to those skilled in the art that other angular increments may be utilized while still permitting implementation of the aforementioned function. It is also clear to those skilled in the art that a variety of press-fit means having projections and apertures may be utilized for the aforementioned function.

In the interchangeable embodiment, the bristle axle 17, which extends through the elongate cylindrical passage 84 of the neck 40, comprises a plurality of complementary splines 44 (best seen in FIGS. 4A-4B). The plurality of complementary splines 44 of the bristle axle 17 of the stem 14 are configured to engage with the series of cooperating splines along the inner surface 60 of the splined cylindrical mandrel 61 of the handle 12. As such, when the stem 14 is attached to the handle 12, and when the motor 58 rotates the drive axle 60, the cylindrical mandrel 61 also rotates and transmits the rotation through its splines 60 to the plurality of complementary splines 44 of the bristle axle 17 and to the rotating bristle assembly 50. Therefore, when the bristle axle 17 is rotated about its longitudinal axis by the rotation means of the handle 14, the rotating bristle assembly 50 also rotates about its longitudinal axis. This feature also can be present in the non-interchangeable embodiment of the toothbrush 10 of a singular bristle axle 17/drive axle 60 configuration is used.

Referring now to FIG. 4C, the interchangeable stem 14, in a second embodiment, is nearly identical to that illustrated in FIGS. 4A and 4B. However, in this embodiment, the interchangeable stem 14 is configured such that the bristle axle 17, attached to the rotating bristle assembly 50, is separable from the rest of the interchangeable stem 14. More specifically, the bristle axle 17 is configured to detachably extend through the elongate cylindrical passage 84 from the bottom of the neck 40 (where the complementary threading 16 is situated) to the top of the neck 40 (where the rotating bristle assembly 50 and the bristle guard 18 are situated). The bristle axle 17 can be pulled out from the elongate cylindrical passage 84 which, in turn, also pulls out the rotating bristle assembly 50. In this way, the second embodiment of the interchangeable stem 14 can have a separately replaceable bristle axle 17 with attached new rotating bristle assembly 50. This allows a user to replace the bristles and attached axle without having to replace the rest of the interchangeable stem 14.

Referring now to FIGS. 6-9, the toothbrush is shown in use. FIG. 6 is a partial perspective view of the electric toothbrush of FIG. 1 operating on the outside facing surfaces of teeth on the upper right side, or the lower left side, of a user's jaw. FIG. 7 is a partial perspective view of the electric toothbrush of FIG. 1 operating on the outside facing surfaces of teeth on the upper left side, or the lower right side, of a user's jaw. FIG. 8 is a partial perspective view of the electric toothbrush of FIG. 1 operating on the inside facing surfaces of teeth on the upper right side, or the lower left side, of a user's jaw. FIG. 9 is a partial perspective view of the electric toothbrush of FIG. 1 operating on the inside facing surfaces of teeth on the upper left side, or the lower right side, of a user's jaw.

In FIGS. 6 and 9, the rotating bristle assembly 17 is spinning in a first direction and in FIGS. 7 and 8, the rotating bristle assembly 17 is spinning in a second direction opposite the rotation of the first direction. More specifically, and as disclosed in more detail below, the rotating bristle assembly should be activated by the user to rotate in a direction such that the bristles always rotate in a direction from the gum to the tooth surface so as to prevent forcing the gums away from the tooth and to prevent forcing debris under the gum between the gum and the tooth surface.

One exemplary method for using the electric toothbrush 10 is as follows. A user grasps the handle 14 of the toothbrush 10 along the outer surface 20. The user positions the handle 14 in the user's grasp such that the user comfortably and easily engages with the activator slide 24. The user can use any physical means within its grasp to engage with the activator slide 24. In the interchangeable embodiment, if the user's personal stem 14 is not already attached to the handle 12, the user then attaches the user's own, personal stem 14 to the handle 12. In the non-interchangeable embodiment, the stem 14 is already permanently attached to the handle 12.

More specifically, in the interchangeable embodiment, the user maneuvers the interchangeable stem 14 (splined end of the bristle axle 17 leading) towards the rotating ring 15 of the handle 12. The user inserts the bristle axle 17 through an opening defined in the top end of the handle 12 such that the plurality of splines 44 extends through the internal cavity 52 of the handle towards the rotation means. The user continues to insert the bristle axle 17 such that the plurality of splines 44 extend into the splined cylindrical mandrel 61 and engage with the series of complementary splines 60 along its internal surface.

In an embodiment with a screw-thread attachment means, the user then comfortably and easily screw threads the stem 14 to the handle 12 via the rotating portion of the rotating ring 15 (the fixed portion of the rotating ring 15 remains secured to the handle 12, which forces the threaded inner surface 32 of the rotating ring 15 to engage with the complementary threaded outer surface 16 of the stem 14). As such, the stem 14 is rigidly retained by the handle 12, and the stem 14 is correctly and fully engaged with the rotation means of the electric toothbrush 10.

With the goal of first cleaning the upper right facial (outer) side and then the lower left facial side, the user slides the activator slide 24 within the channel 26 of the handle 12 to the second “on” position (best seen in FIG. 6). This turns “on” the electric toothbrush 10, which drives counterclockwise rotation of the rotating bristle assembly 50 of the stem 14. More specifically, the battery 56 powers the motor 58, which drives rotation of the drive axle 60, which in turn drives rotation of the splined cylindrical mandrel 61, which in turn drives rotations of the bristle axle 17 which in turn drives rotation of the rotating bristle assembly 50.

The user then maneuvers the stem 14 of the electric toothbrush 10 into the user's mouth towards the upper right facial side of the jaw 70. The user applies the counterclockwise rotating rotating bristle assembly 50 to the teeth starting along the gingival margin and works from the gum towards the tooth crown. The user does this to every tooth in the upper right facial side of the jaw 70. When the user finishes brushing the upper right facial side of the jaw 70, the user makes a series of comfortable and easy wrist pronations and is in a position to brush the teeth on the lower left facial side of the jaw 70. More specifically, the user rotates the toothbrush approximately 180 degrees, which puts the toothbrush 10 in a more or less proper position for brushing the teeth on the lower left facial side of the jaw 70. The user then maneuvers the stem 14 of the toothbrush 10 towards the lower left facial side of the jaw 70. The user applies the counterclockwise rotating rotating bristle assembly 50 to the teeth starting along the gingival margin and works towards the tooth crown. The user does this to every tooth in the lower left facial side of the jaw 70.

Then, with the goal of cleaning the upper left facial side and the lower right facial side, the user slides the activator slide 24 of the handle 12 to the first “on” position (best seen in FIG. 7). This turns “on” the electric toothbrush, which drives clockwise rotation of the rotating bristle assembly 50 of the stem 14. The user then maneuvers the stem 14 of the toothbrush 10 towards the upper left facial side of the jaw 70. The user applies the clockwise rotating rotating bristle assembly 50 to the teeth starting along the gingival margin and works towards the tooth crown. The user does this to every tooth in the upper left facial side of the jaw 70. When the user finishes brushing the upper left facial side of the jaw 70, the user makes a series of comfortable and easy wrist pronations and is in a position to brush the teeth on the lower right facial side of the jaw 70. More specifically, the user rotates the toothbrush approximately 180 degrees, which puts the toothbrush 10 in a more or less proper position for brushing the teeth on the lower right facial side of the jaw 70. The user then maneuvers the stem 14 of the toothbrush 10 towards the lower right facial side of the jaw 70. The user applies the counterclockwise rotating rotating bristle assembly 50 to the teeth starting along the gingival margin and works towards the tooth crown. The user does this to every tooth in the lower right facial side of the jaw 70.

Then, with the goal of cleaning the upper right lingual (inner) side and the lower left lingual side (best seen in FIG. 8), the user again makes a series of comfortable and easy wrist pronations and maneuvers the stem 14 towards the upper right lingual side of the jaw 70. The user applies the clockwise rotating rotating bristle assembly 50 to the teeth starting along the gingival margin and works towards the tooth crown. The user does this to every tooth in the upper right lingual side of its jaw 70. When the user finishes brushing the upper right lingual side of the jaw 70, the user again makes a series of comfortable and easy wrist pronations and maneuvers the stem 14 towards the lower left lingual side. The user applies the clockwise rotating rotating bristle assembly 50 to the teeth starting along the gingival margin and works towards the tooth crown. The user does this to every tooth in the lower left lingual side of its jaw 70.

Then, with the goal of cleaning the upper left lingual side and the lower right lingual side, the user slides the activator slide 24 of the handle 12 to the second “on” position (best seen in FIG. 9). This turns “on” the electric toothbrush, which drives counterclockwise rotation of the rotating bristle assembly 50 of the stem 14. The user then maneuvers the stem 14 of the toothbrush 10 towards the upper left facial side of the jaw 70. The user applies the counterclockwise rotating rotating bristle assembly 50 to the teeth starting along the gingival margin and works towards the tooth crown. The user does this to every tooth in the upper left facial side of its jaw 70. When the user finishes brushing the upper left lingual side of the jaw 70, the user again makes a series of comfortable and easy wrist pronations and maneuvers the stem 14 towards the lower right lingual side. The user applies the counterclockwise rotating rotating bristle assembly 50 to the teeth starting along the gingival margin and works towards the tooth crown. The user does this to every tooth in the lower right lingual side of the jaw 70.

Then, the user can brush the outer and inner surfaces of the front top and bottom teeth in much the same manner, taking care to have the rotating bristle assembly 50 rotating in the proper direction from the gum to the tooth surface. Finally, the user can brush the biting occlusal surfaces of the teeth, specifically the occlusal surfaces of the molars, using the rotating bristle assembly 17 in either the clockwise or counterclockwise direction as no gum contact is involved.

Having brushed the periodontium, and all the tooth surfaces, while essentially maintaining the same grip on the handle 12 and generally only having to use one arm, the user slides the activator slide 24 of the handle 12 to the intermediate position. This turns “off” the electric toothbrush, which stops rotation of the rotating bristle assembly 50 of the stem 14.

If the toothbrush 10 is provided with a suitable outer surface to the bristle guard 18, and if the user desires to brush the tongue, inner cheeks, or gums, the user then can make a series of comfortable and easy wrist pronations to rotate the bristle guard 18 to face the tongue, inner cheek, or gums. The user then maneuvers the stem 14 towards the tongue, inner cheek, or gums and scrubs the tongue, inner cheek, or gums with the outer surface 19 of the bristle guard 18. The user preferably does this to the entire surface of the tongue, inner cheek, and/or gums. Having brushed the entire oral cavity, while essentially maintaining the same grip on the handle 12, the user removes the stem 14 of the toothbrush 10 out of the mouth and places the toothbrush 10 down.

Referring now to FIGS. 10A and 10B, an electric toothbrush of the present invention may comprise a second embodiment of a handle and a bristle shield. The second embodiment of the handle 12 comprises a knurled threaded cap 300 instead of the snap fit lid 100 to access the battery 52. The knurled threaded cap 300 is configured to be water-tight and easy to open.

The bristle shield 200 is configured as an elongate cylindrical domed cap 200 that simply goes over and surrounds the interchangeable stem 14. The interchangeable stem 14 would be inserted into the interior spaced defined by the elongate cylindrical domed cap 200. The end rim of the elongate cylindrical domed cap 200 press-fits up against the end of the handle 12, proximate to the interchangeable stem 14, and securely engages onto the handle 12. It is envisioned that the end of the handle 12 proximate to the interchangeable stem 14 may be slightly tapered and/or surfaced textured to facilitate and improve the press-fit with the rim of the elongate cylindrical domed cap 200.

As such, the bristle shield 200 provides a built-in feature for additional protection against bristle-introduced infections to the periodontium as the bristles 53 can be shielded from the bathroom air. Moreover, as the bristle shield 200 extends out around the previously unbound portion of the bristles 53, the bristles shield 200 allows sufficient air around the bristles 53 so as to appropriately dry between tooth brushings.

Referring now to FIGS. 11A, 11B, and 11C, a first exemplary embodiment of the electric toothbrush of the present invention is video capable and specialized for proper periodontium prophylaxis. The video capable embodiment is identical to the electric toothbrush embodiment of FIGS. 10A and 10B except for the following description.

The video capable embodiment comprises a handle body 12, defining a cylindrical body, and an interchangeable stem 14, having a head 40 with a rotating bristle assembly 50. The video capable embodiment also comprises a bristle shield 200 defining an elongate cylindrical domed cap for the interchangeable stem 14. The video capable embodiment also comprises a camera device 500 communicatively coupled, via a pathway 600, to a user useful display 700. In this way, the video capable embodiment is configured to provide a user with live intraoral video obtained during operation of the electric toothbrush.

The handle body 12 comprises a switch means for activating and deactivating 24 (turning on and off) an internal motor 58, a battery 56, electrical components for powering the rotating bristle assembly 50, and a knurled threaded cap 300 to access the battery 56 and/or any other internal components. The handle body 12 is configured to provide a suitable handgrip for a user about the cylindrical body. The cylindrical body also defines a means for detachably engaging 400 the camera device 500 to an exterior side of the electric toothbrush.

In the exemplary embodiment of FIGS. 11A, 11B, and 11C, the means for detachably engaging 400 is a two part press fit system (400a and 400b) configured to tightly receive the camera device 500. The camera device 500 may be rigidly but removably retained between the means for detachably engaging 400a and 400b such that a user gripping the handle body 12 does not obstruct a video feed of his/her gingival margin while using the electric toothbrush 10. Because the appropriate daily prophylaxis accomplished by the electric toothbrush 10 requires minimal toothpaste (and the foam created therefrom), the video capable embodiment is characterized by unobstructed video imaging. Moreover, the means for detachably engaging 400a and 400b is substantially antipodal to the switch means 24; although, various positions about the exterior of the handle body 14 are envisioned.

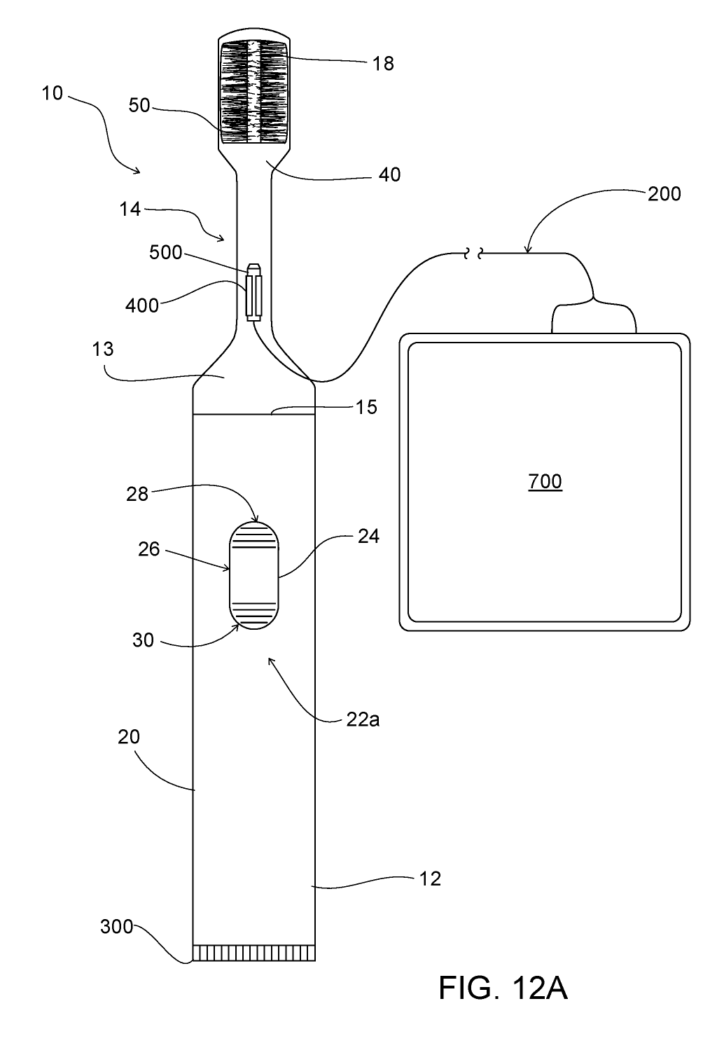

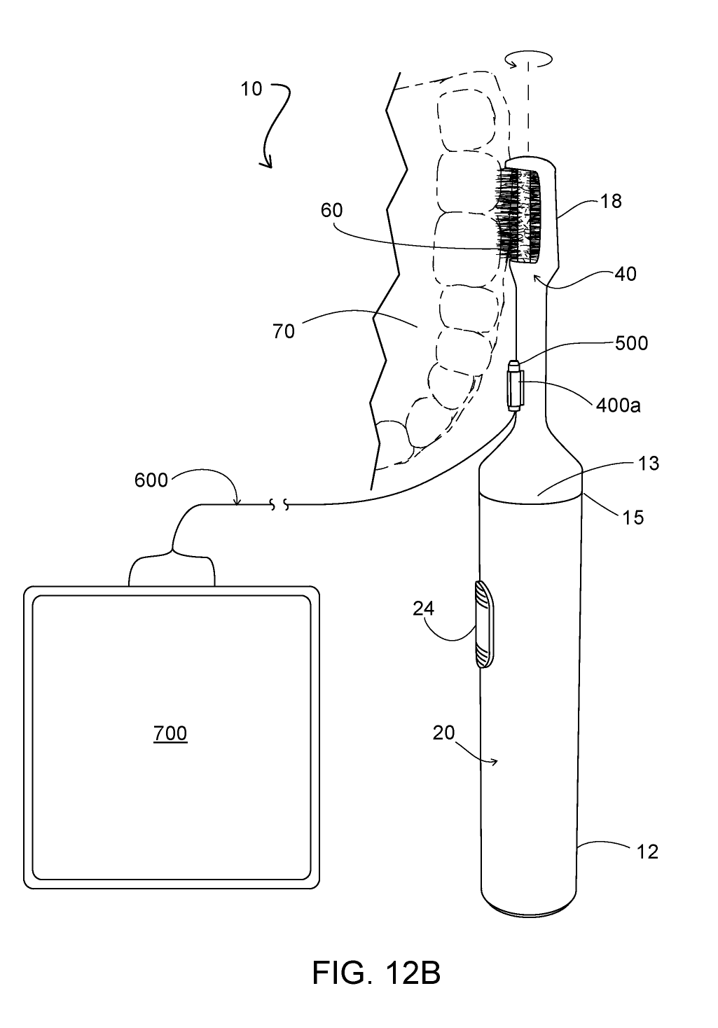

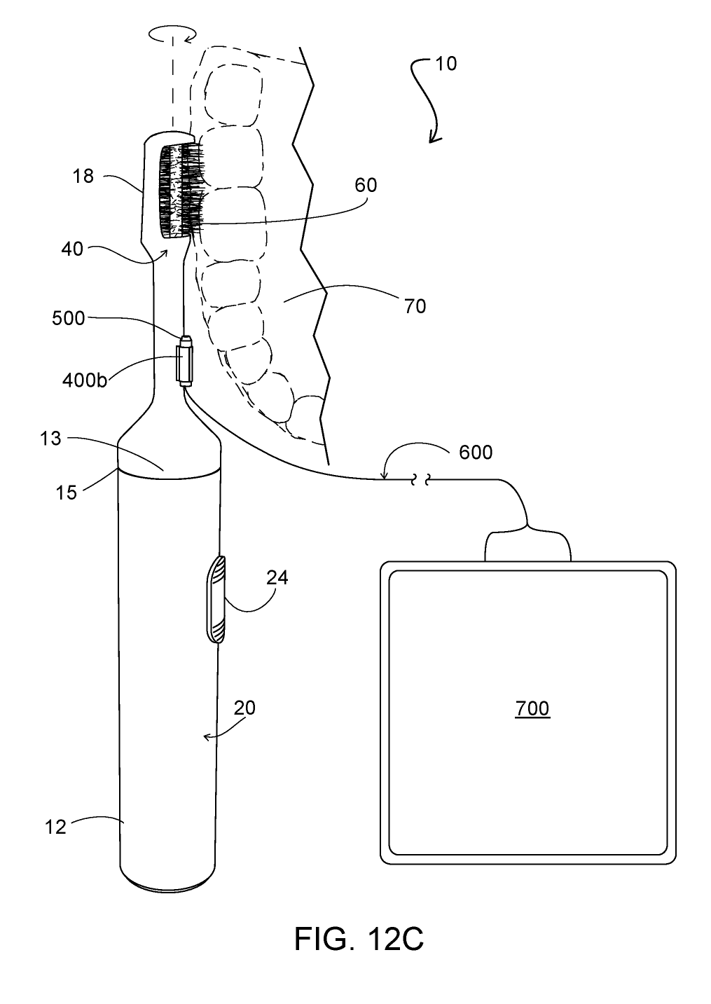

In certain exemplary embodiments, the means for detachably engaging 400 may be an integral part of the handle body 12 or may be a separate and distinct component attached thereto. Moreover, the means for detachably engaging 400 is not limited to positioning on and/or orientation relative to the handle body 12. A person having ordinary skill in the art understands that the means for detachably engaging 400 may be part of/attached to the interchangeable stem 14 or any other component of the electric toothbrush 10 (see FIG. 12).

Moreover, the means for detachably engaging 400 may be larger/smaller and structured according to the embodiments of the camera device 500. In this way, the means for detachably engaging 400 may be structured to complement and/or conform to the camera device 500. Moreover, the means for detachably engaging 400 is not limited to a friction fit engagement. A person having ordinary skill in the art understands that the means for detachably engaging 400 may incorporate magnets, clips, protrusions, surface features, etc. for retaining the camera device 500 in position, alignment, and orientation.

In the exemplary embodiment of FIGS. 11A, 11B, and 11C, the camera device 500 is a flexible borescope/optical device consisting of a flexible conductive cable (optionally, tubular), a lens, a camera sensor, a light mechanism, and a relay optical system as is understood by a person having ordinary skill in the art. The aperture and lighting mechanism of the camera device 500, when detachably engaged to the handle body 12, is aimed towards the oral cavity of a user so as to capture intraoral video. The intraoral video is then transmitted, via the pathway 600, to the user useful display 700. The lighting mechanism can be six individual LED lights surrounding the aperture of the camera device 500; although, other lighting mechanisms known to a person having ordinary skill in the art are envisioned.

In certain exemplary embodiments, the camera device 500 may be a Depstech™ Micro USB borescope waterproof inspection camera or any other equivalent device known to a person having ordinary skill in the art. For these embodiments, the flexible tube/tubular conductive cable is the pathway 600 communicatively coupling the camera device 500 to the user useful display 700. The camera device 500 is not limited to this specific borescope embodiment. A person having ordinary skill in the art understands that the camera device may include many variations and combinations of articulation mechanism components, ranges of articulation, fields of view, and angles of view as well as any known and applicable objective lens and/or digital camera sensors. Moreover, the camera device 500 may include 10,000 pixels to 22,000 pixels. Moreover, the camera device 500 may be configured for 640×480 resolution, or upwards of 4K HD video, or any other video capture capability known to a person having ordinary skill in the art.

In certain exemplary embodiments, the camera device 500 may be a self-contained optical device comprising a power source, wiring, electronic boards/circuitry, system bus, and/or means for capturing, processing, and/or transmitting video data for use, directly or indirectly, by a user. In this way, the camera device 500 may be configured to capture, process, and transmit an intraoral video feed, via a USB/power cable 600, to a user useful display 700. The user useful display 700 may be a 3-inch LCD display with 320×240 pixels or better, or any other equivalent or upgraded embodiment known to a person having ordinary skill in the art.

Moreover, the camera device 500 may be a multi-component system having the components and means for processing and/or transmitting video data spread out across the electric toothbrush 10. For example, in one exemplary embodiment, the power source, electronic boards/circuitry, system bus, and means for capturing, processing, and/or transmitting video data is housed within the handle body 14 and/or the user useful display 700.

In another exemplary embodiment, the system components may be spread out between the camera device 500 and the user useful display 700. In these embodiments, the camera device 500 may be configured to capture a video feed and/or partially process the video feed for transmission via a wireless communications pathway 600 to the user useful display 700. A person having ordinary skill understands that the wireless communications pathway 600 may be Bluetooth® enabled, cellular telecommunications network enabled, Wi-Fi® enabled, etc. The user useful display 700 may also be configured to receive and/or partially process the video feed for display to the user. A person having ordinary skill understands that the user useful display 700 may be a portable computing device/smart phone configured to process video data, decrease video distortion, and/or improve video quality. For example, in one exemplary embodiment, the user useful display 700 is an USB OTG (on-the-go) compatible Android® smartphone; however, many other similar devices are envisioned.

Referring now to FIGS. 12A, 12B, and 12C, a second exemplary embodiment of a video capable electric toothbrush of the present invention is shown. This second video capable embodiment is identical to the first video capable electric toothbrush embodiment of FIGS. 11A, 11B, and 11C except for the following description.

The second video capable embodiment comprises the means for detachably engaging 400, as a two part press fit system (400a and 400b), but has it positioned at the elongated neck of the interchangeable stem 14. The camera device 500 may be rigidly but removably retained between the means for detachably engaging 400a and 400b such that a user inserting the elongate neck into his/her oral cavity does not obstruct the video feed of his/her gingival margin while using the electric toothbrush 10. Because the appropriate daily prophylaxis accomplished by the electric toothbrush 10 requires minimal toothpaste (and the foam created therefrom), the second video capable embodiment is also characterized by unobstructed video imaging. Moreover, the means for detachably engaging 400a and 400b is aligned to the switch means 24.

In certain exemplary embodiments, the means for detachably engaging 400 may be an integral part of the interchangeable stem 14 or may be a separate and distinct component attached thereto. Moreover, the means for detachably engaging 400 is not limited to positioning on and/or orientation onto the elongate neck. A person having ordinary skill in the art understands that the means for detachably engaging 400 may be part of/attached to the any portion of component of the interchangeable stem 14.

In the exemplary embodiment of FIGS. 12A, 12B, and 12C, the camera device 500 is also a flexible borescope/optical device consisting of a flexible conductive cable (optionally, tubular), a lens, a camera sensor, a light mechanism, and a relay optical system as is understood by a person having ordinary skill in the art. The aperture and lighting mechanism of the camera device 500, when detachably engaged to the interchangeable stem 14, is aimed towards the oral cavity of a user so as to capture intraoral video. The intraoral video is then transmitted, via the pathway 600, to the user useful display 700.

The electric toothbrush for proper periodontium prophylaxis may be marketed in a kit form with a single handle 12 and a plurality of interchangeable stems 14. The various stems 14 can thus be replacements, specific task oriented shapes/stiffness, or individually assigned to different members of a family. Color distinction and firmness are thus often characteristics of the different heads in a kit. Although handle 12 is discussed as preferably battery powered, of course, the invention also includes other well-known power supplies such as corded for outlet connection or rechargeable batteries and an associated brush holder/charger.

Although the particular embodiments shown and described above will prove to be useful in many applications in the electric toothbrush art to which the present invention pertains, further modifications of the present invention will occur to persons skilled in the art. All such modifications are deemed to be within the scope and spirit of the present invention as defined by the appended claims.

What is claimed is:

1. A video capable electric toothbrush system for proper periodontium prophylaxis, the electric toothbrush system comprising:
a) a video display;
b) a camera device communicatively coupled, via a communications pathway, to the video display, the camera device configured to capture and transmit an intraoral video feed for display at the video display;
c) a handle body; and
d) an interchangeable stem comprising an elongate neck, a head, a rotating bristle assembly, a means for rotating the rotating bristle assembly, and a bristle guard, the elongate neck comprising a first end opposite a second end, wherein (i) the first end of the elongate neck is configured to detachably engage with the handle body, (ii) the second end of the elongate neck is terminated by the head, (iii) the head comprises a rotating bristle assembly longitudinally aligned with a longitudinal axis of the interchangeable stem, (iv) the rotating bristle assembly comprises a first end opposite a second end, (v) the first end of the rotating bristle assembly is proximate to the second end of the elongate neck, and (vi) the bristle guard extends from the second end of the neck around the side of the rotating bristle assembly;
wherein the video display is remote from an assembly of the handle body, the interchangeable stem, and the camera;
wherein the camera device of the assembly is positioned and aimed towards the rotating bristle assembly so as to capture a visual field about the rotating bristle assembly;
wherein the longitudinal axis of the bristle guard is parallel to the longitudinal axis of the interchangeable stem;
wherein the bristle guard is laterally spaced away from the rotating bristle assembly;
wherein the bristle guard bounds an arc segment along the rotating bristle assembly; and
wherein the means for rotating the rotating bristle assembly is powered and the rotating bristle assembly is selectably motorized to rotate clockwise or counterclockwise via mechanical engagement with the means for rotating the rotating bristle assembly.

2. The electric toothbrush of claim 1, wherein the video display comprises electronic boards and circuitry for receiving and at least partially processing the intraoral video feed transmitted by the camera device.

3. The electric toothbrush of claim 1, wherein the video display is at least one of a portable computing device and a smartphone.

4. The electric toothbrush of claim 1, wherein the camera device defines an aperture and comprises a lens, a digital camera sensor, a lighting mechanism, and a relay optical system, the camera device having a video capture capability.

5. The electric toothbrush of claim 4, wherein the lighting mechanism comprises one or more individual light emitting diodes positioned proximate to the aperture so as to illuminate the visual field about the rotating bristle assembly.

6. The electric toothbrush of claim 1, wherein the camera device comprises electronic boards and circuitry for transmitting and at least partially processing the intraoral video feed before transmitting the intraoral video feed to the video display.

7. The electric toothbrush of claim 1, wherein the handle body defines a means for detachably engaging the camera device to an exterior side of the handle body so as to position and aim the camera device towards the visual field about the rotating bristle assembly.

8. The electric toothbrush of claim 7, wherein:
the handle body additionally comprises the means for rotating the rotating bristle assembly; and
the means for rotating the rotating bristle assembly comprises a bristle axle mechanically engaged with the rotating bristle assembly, the bristle axle extending through the elongate neck, the bristle axle configured to rotate clockwise or counterclockwise the rotating bristle assembly.

9. The electric toothbrush of claim 8, wherein one end of the bristle axle is engaged to one end of the bristle guard, the bristle axle configured to rotate about the end of the bristle guard as the bristle axle rotates clockwise or counterclockwise.

10. The electric toothbrush of claim 1, wherein the bristle guard comprises a bristle-facing side and a non-bristle facing side, the non-bristle facing side defining a raised surface texture or raised surface feature that is configured for tongue cleaning.

11. The electric toothbrush of claim 1, wherein the bristle guard is configured to incrementally rotate around the rotating bristle assembly.

12. A video capable electric toothbrush for proper periodontium prophylaxis, the electric toothbrush comprising:
a) a video display integral to one of a portable computing device and a smartphone, the one of a portable computing device and a smartphone comprising electronic boards and circuitry for receiving and at least partially processing an intraoral video feed;
b) a camera device communicatively coupled, via a communications pathway, to the video display, the camera device configured to capture and transmit the intraoral video feed for display at the video display;
c) a handle body defining a means for detachably engaging the camera device to an exterior side; and
d) an interchangeable stem comprising an elongate neck, a head, a rotating bristle assembly, and a means for rotating the rotating bristle assembly, and a bristle guard, the elongate neck comprising a first end opposite a second end, the bristle guard comprising a bristle-facing side and a non-bristle facing side, the non-bristle facing side defining a raised surface texture or raised surface feature that is configured for tongue cleaning, wherein (i) the first end of the elongate neck is configured to detachably engage with the handle body, (ii) the second end of the elongate neck is terminated by the head, (iii) the head comprises a rotating bristle assembly longitudinally aligned with a longitudinal axis of the interchangeable stem, (iv) the rotating bristle assembly comprises a first end opposite a second end, (v) the first end of the rotating bristle assembly is proximate to the second end of the elongate neck, and (vi) the bristle guard extends from the second end of the neck around the side of the rotating bristle assembly;
wherein the video display is remote from an assembly of the handle body, the interchangeable stem, and the camera;
wherein the camera device is positioned and aimed on the handle body towards the rotating bristle assembly so as to capture a visual field about the rotating bristle assembly;
wherein the longitudinal axis of the bristle guard is parallel to the longitudinal axis of the interchangeable stem;
wherein the bristle guard is laterally spaced away from the rotating bristle assembly;
wherein the bristle guard bounds an arc segment along the rotating bristle assembly; and
wherein the longitudinal axis of the interchangeable stem is substantially aligned with a longitudinal axis of the handle body, the means for rotating the rotating bristle assembly is powered, and the rotating bristle assembly is selectably motorized to rotate clockwise or counterclockwise via mechanical engagement with the means for rotating the rotating bristle assembly.

13. The electric toothbrush of claim 12, wherein the camera device defines an aperture and comprises a lens, a digital camera sensor, a lighting mechanism, and a relay optical system.

14. The electric toothbrush of claim 13, wherein the lighting mechanism comprises one or more individual light emitting diodes positioned proximate to the aperture so as to illuminate the visual field about the rotating bristle assembly.

15. The electric toothbrush of claim 12, wherein the camera device comprises electronic boards and circuitry for transmitting and at least partially processing the intraoral video feed before transmitting the intraoral video feed to the one of a portable computing device and a smartphone.

16. A video capable electric toothbrush for proper periodontium prophylaxis, the electric toothbrush comprising:
a) a video display;
b) a flexible borescope communicatively coupled, via a communications pathway, to the video display, the flexible borescope configured to capture and transmit an intraoral video feed for display at the video display;
c) a handle body defining a means for detachably engaging the flexible borescope to an exterior side, the means for detachably engaging configured to at least partially complement and conform to the flexible borescope; and
d) an interchangeable stem comprising an elongate neck, a head, a rotating bristle assembly, and a means for rotating the rotating bristle assembly, and a bristle guard, the elongate neck comprising a first end opposite a second end, the bristle guard comprising a bristle-facing side and a non-bristle facing side, the non-bristle facing side defining a raised surface texture or raised surface feature that is configured for tongue cleaning, wherein (i) the first end of the elongate neck is configured to detachably engage with the handle body, (ii) the second end of the elongate neck is terminated by the head, (iii) the head comprises a rotating bristle assembly longitudinally aligned with a longitudinal axis of the interchangeable stem, (iv) the rotating bristle assembly comprises a first end opposite a second end, (v) the first end of the rotating bristle assembly is proximate to the second end of the elongate neck, and (vi) the bristle guard extends from the second end of the neck around the side of the rotating bristle assembly;
wherein the video display is remote from an assembly of the handle body, the interchangeable stem, and the camera;
wherein the flexible borescope is positioned and aimed on the handle body towards the rotating bristle assembly so as to capture a visual field about the rotating bristle assembly;
wherein the longitudinal axis of the bristle guard is parallel to the longitudinal axis of the interchangeable stem;
wherein the bristle guard is laterally spaced away from the rotating bristle assembly;
wherein the bristle guard bounds an arc segment along the rotating bristle assembly; and
wherein, when the interchangeable stem is detachably engaged with the handle body:
the longitudinal axis of the interchangeable stem is substantially aligned with a longitudinal axis of the handle body;
the means for rotating the rotating bristle assembly is powered; and
the rotating bristle assembly is selectably motorized to rotate clockwise or counterclockwise via mechanical engagement with the means for rotating the rotating bristle assembly.

17. The electric toothbrush of claim 16, wherein:
the video display is integral to one of a portable computing device and a smartphone, the one of a portable computing device and a smartphone comprising electronic boards and circuitry for receiving and at least partially processing the intraoral video feed transmitted by the flexible borescope; and
the flexible borescope comprises electronic boards and circuitry for transmitting and at least partially processing the intraoral video feed before transmitting the intraoral video feed to the video display.

18. The electric toothbrush of claim 16, wherein:
the flexible borescope is a USB-enabled waterproof borescope with at least one of a lighting mechanism and an articulation mechanism, wherein the communications pathway is at least partially a USB cable configured to power the flexible borescope; and
the video display is a USB on-to-go compatible device.

19. The electric toothbrush of claim 18, wherein the lighting mechanism comprises one or more individual light emitting diodes positioned proximate to an aperture of the flexible borescope so as to illuminate the visual field about the rotating bristle assembly.

20. The electric toothbrush of claim 16, wherein the means for detachably engaging the flexible borescope to an exterior side of the handle body is a two-part press fit system configured to tightly receive between the two parts, via a friction fit, the flexible borescope so as to position and aim the flexible borescope towards the visual field about the rotating bristle assembly.
